(19) United States
(12) Patent Application Publication
(10) Pub. No.: US 2010/0312176 A1
(43) Pub. Date: Dec. 9, 2010

(54) SYSTEM AND METHOD FOR MONITORING AND REGULATING BLOOD GLUCOSE LEVELS

(75) Inventors: Hans-Martin Lauer, Munchen (DE); Matthias Wufka, Munchen (DE); Sebastian Hörnig, Karlsfeld (DE); Doris Röthlein, Kassel (DE)

Correspondence Address: RATNERPRESTIA, P.O. BOX 980, VALLEY FORGE, PA, 19482, US

(73) Assignee: B. BRAUN MELSUNGEN AG, MELSUNGEN (DE)

(21) Appl. No.: 12/681,317

(22) PCT Filed: Oct. 1, 2008
(86) PCT No.: PCT/EP2008/063122

§ 371 Date: Aug. 20, 2010

(30) Foreign Application Priority Data
Oct. 2, 2007 (DE) 10 2007 047 351.8

(51) Int. Cl. A61M 5/14

(52) U.S. Cl. 604/66

(57) ABSTRACT

A system and method for monitoring and regulating blood glucose values in blood circulation. The system includes an input device for receiving at least one blood glucose value measured in the blood circulation, at least one insulin value previously supplied to the blood circulation by at least one insulin supply device, and/or at least one nutrition value of at least one artificial nutrition agent previously supplied to the blood circulation by at least one nutrition supply device. The system also includes a computer for calculating a new insulin value and optionally a new nutrition value according to the influence thereof on blood glucose values and according to the previously measured blood glucose value, and an output device for outputting the new insulin values and optionally nutrition values.

### CROSS REFERENCE TO RELATED APPLICATIONS

This application is the U.S. national phase application of PCT International Application No. PCT/EP2008/063122, filed Oct. 1, 2008, which claims priority to German Patent Application No. DE 10 2007 047 351.8, filed Oct. 2, 2007, the contents of such applications being incorporated herein by reference.

### FIELD OF THE INVENTION

The invention relates to a system and a method for monitoring and regulating blood glucose levels in the blood stream.

### BACKGROUND OF THE INVENTION

An example of a system for monitoring the blood glucose level in the body tissue of a patient is known from EP 0 461 207 B1, which system is directed to detect, by means of implantable glucose-sensitive living cells capable of producing an electrical or optical signal in response to the glucose concentration in the medium, the electrical and optical signals by way of means located outside the living cells. Because of the required implantation, such cells are less suitable for use in patients lying on an intensive care unit.

From EP 1 185 321 B1, an infusion system having a closed-loop control circuit for infusing a fluid in a consumer is known, wherein a sensor system monitors the glucose concentration and the sensor signal output by said system generates a controller input which is used by a controller for generating commands which are forwarded to a delivery system, e.g. for insulin. This controller is a specific proportional-integral-differential controller. Any values exceeding the actual glucose levels, which might have an effect on the glucose level, will be considered by this system, if at all, only to a minor degree.

The EP 1 458 435 B1 describes a system for dispensing medicaments using an infusion pump and a controller including an algorithm for controlling the delivery of medicaments by the infusion pump. The controller includes a plurality of medicament delivery profiles for delivering a medicament from the storage container. The controller shows a plurality of suspend functions, wherein at least one of the plurality of medicament delivery profiles may be separately and temporarily suspended. It is possible for a first delivery profile for delivering a first medicament to be suspended, whilst the second delivery profile for delivering a second medicament continues. In this respect the emphasis is placed on a combination of a plurality of delivery profiles, whilst the insulin factor, which influences the glucose level, is considered. A control of medication as may be suitable for a patient lying on an intensive care unit and needing artificial feeding is not described here.

### SUMMARY OF THE INVENTION

In at least one aspect, the present invention provides a system and a method for monitoring and regulating blood glucose levels in a blood stream, wherein monitoring and regulating is possible also in the case of the blood stream of a patient lying on an intensive care unit and thus being in need of artificial feeding, as a function of the parameters of the artificial nutrition which influence the glucose level.

An aspect of at least one embodiment of the invention lies in the fact that in a system for monitoring and regulating blood glucose levels in a blood stream, the following components are provided:
an input unit for receiving at least one measured blood glucose level present in the blood stream, at least one insulin level so far supplied to the blood stream by means of at least one insulin supply unit, and/or at least one nutritional value, if applicable, of artificial nutrition so far supplied to the blood stream by means of at least one nutrition supply unit,
a calculation unit for calculating a new insulin value or a new insulin rate and, if applicable, a new nutritional value as a function of its effect on the blood glucose levels and as a function of the blood glucose level previously measured, and
an output unit for outputting the new insulin values or the new insulin rate and, if applicable, a nutritional value which may be supplied via an enteral or a parenteral route.

A method according to at least one embodiment of the invention for monitoring and regulating blood glucose levels in a blood stream comprises the following steps:
starting at least one nutrition supply unit for directly or indirectly supplying preferably artificial nutrition into the blood stream,
determining at least one nutritional value of the preferably artificial nutrition from a plurality of data items transferred from the nutrition supply unit to a control unit,
transferring measured blood glucose levels present in the blood stream to the control unit,
calculating an insulin value or an insulin rate and, if applicable, a nutritional value as a function of its effect on blood glucose levels and as a function of the previously measured blood glucose level by means of a calculation unit, and
outputting, the calculated new insulin values or insulin rate and, if applicable, the calculated new nutritional values by means of an output unit.

This means that data regarding nutritional values of a parenteral and/or enteral feeding, blood glucose levels, patient data such as weight information and the like, and insulin values may be input into the calculation unit as input data. As output data, insulin values or a new insulin rate, the time of the next measurement, any glucose plausibility check values and, if applicable, nutritional values are output by the calculation unit.

Particularly the time of the next measurement is to be seen as an important output value and here a time interval of 0.5 to 4 hours since the last measurement is preferably used. If such a measurement does not take place within the indicated time interval, staff will be alerted.

Such a system and such a method may advantageously be used with patients who are lying on an intensive care unit and rely on artificial feeding, without any risk of over- or under-dosing of the amount of insulin supplied developing as a result of a neglect of the influencing factors by the artificial nutrition supplied. The blood stream of a person can evoke rapid reactions to incorrect insulin rates and may therefore, if insulin is administered without or with only an insufficient supply of nutrition at the same time, fall into a life-threatening hypoglycaemic condition. In this connection it is to be considered that the nutrition supplied to the patient has parameters which are relevant to the blood sugar level, such as in particular the carbohydrate concentration of the nutrition and the amount of carbohydrates continuously administered in terms of carbohydrates per time unit (carbohydrate rate) or as a bolus.

If such carbohydrate values are taken into account in the calculation of a new insulin value, an interdependent consideration of the nutritional values, the insulin values and the blood glucose levels resulting therefrom will take place, which may avoid any undesired reactions of the human body to substances supplied to it. At the same time, the amount and the duration of the insulin supplied may be calculated as a function of the circumstance as to whether the human body is supposed to be fed with nutrition having changed nutritional values, so that an adaptation to the type of the artificial nutrition supplied, i.e. the type of nutrition, may be carried out when the insulin values are being calculated. It is possible to carry out both enteral and parenteral feeding.

Similarly, it may be taken into account in such a system or method that the artificial feeding is suspended, which has an effect on the blood glucose level. The fact that the insulin rate will be newly calculated as a result of this means that the negative effect caused by this suspension of the artificial feeding may be compensated, so that no overreaction of the blood stream due to any imminent disadvantageous glucose values will take place. In the case of a feeding stop, for example by interrupting the parenteral or enteral feeding, whilst the supply of insulin is continued at the same insulin rate, there will be a risk of hypoglycaemia due to an insulin overdose.

The artificial feeding may be carried out both continuously and in a bolus-type manner and may be supplied in an automated manner as a response to the previously calculated new nutritional values and/or new insulin values or manually, by operating personnel reading the previously calculated values from a display device or specifying values they generated themselves. The main priority however is to determine insulin values or an insulin rate on the basis of the values previously input into the calculation unit with regard to nutrition and blood glucose.

Of course, the nutritional values of supplied nutrition may also be modified independently from calculated values on the basis of an individually desired change of nutrition. In this respect, the artificial feeding is to be regarded as an input value into the calculation unit, which in turn causes a new insulin value or a new insulin rate to be output as an output value under consideration of the blood glucose level.

The input and output units are combined in a control unit having a transmission and reception unit. Such a control unit (Space Control) is in direct communication with the calculation unit (Space Com). Alternatively, both units may be integrated in a common device.

The output unit is connected to the display unit, which first and foremost is used for displaying and reading off newly calculated insulin values and, if applicable, nutritional values and, if applicable, also for providing at the same time an input unit in the form of a touch screen, via which the measured blood glucose levels and, if applicable, any desired new nutritional values and/or new insulin values are input into the system. Such a user interface is in direct communication with a therapy control unit which is mounted in the control unit and is responsible for the control and exchange of data transmitted from and to the calculation unit.

The output unit is preferably coupled to the nutrition supply unit and the insulin supply unit in cases where an automated supply of insulin and artificial nutrition with the newly calculated insulin values and nutritional values is supposed to be carried out.

According to a preferred embodiment, a polling signal for polling the nutritional values is transmitted from the control unit to the nutrition supply unit at least at one predeterminable point in time. This polling signal is used to communicate the polled nutritional values, for example the current delivery rate of the nutrition supply unit implemented as a pump, the type of nutrition, which means the nutrition medicament which is administered and the carbohydrate concentration thereof, to the control unit to enable it to calculate from this data a carbohydrate rate as the relevant nutritional value. Subsequently, the carbohydrate rate is transferred to the calculation unit for calculating the new insulin values or the new nutritional values.

Instead of the display unit in the form of a touch screen as described above, which at the same time allows the various data such as for example the measured blood glucose data or further patient-specific values to be input by the operator, a separate input unit for inputting the measured or calculated blood glucose levels and/or nutritional values and further patient-specific values such as body weight, age of the person and similar data may be used as an input unit. Examples of this are conventional keyboards or operable cursor control devices such as, for example, a mouse.

The essential values which are input via such an input unit and which are relevant for calculating the new insulin value or the new insulin rate and, if applicable, new nutritional values, are the blood glucose value which may be measured by means of a separate device in the blood stream, for example of a patient, or outside of it, any change to the artificial nutrition, which will result in a desired modification of the nutritional values, and/or a change of the patient's weight. The input of this data or of these values will in any case result in a calculation of new insulin values or insulin rates and, if applicable, of new nutritional values, in order to obtain in this way an adaptation of the blood stream to the modified data and a new adjustment of the interdependence of insulin values, blood glucose levels and nutritional values. Of course, in the case of for example a desired change of the nutritional values which may have been made by an operator and which will then be made known to the system via the input unit, not a new nutritional value but only a new insulin value will be calculated.

According to a preferred embodiment, an alarm unit is provided in the control unit for visually and/or acoustically alerting an operator that it is time to take a new measurement of the blood glucose level. Such an alarm unit may also, or in addition, trigger an alarm in the case of an unwanted stop of the artificial feeding process for example due to a technical failure. Also, an alarm may be triggered in the case of any undesired change of nutrition.

The undesired changes to the feeding that will cause an alarm to be triggered can be categorised into three different scenarios: there may be an undesirable interruption of the feeding process, which may be both an enteral and a parenteral feeding. Alternatively, the rate of the enteral or parenteral feeding may be changed. A third possibility would be a change in nutrition when a disposable article of the nutrition supply unit is replaced.

An alarm is triggered in intervals of approximately five minutes, preferably five minutes after a change of nutrition, unless an input for calculating the new insulin rate has been made into the control unit or the input unit in the meantime.

An alarm indicating that it is time for the blood glucose level to be taken may be carried out for example according to the following procedure: a pre-alarm is raised 10 minutes prior to the actual due time of the next measurement. A time interval for measurements to be taken may be, for example, in a range of 0.5-4 hours. Such an alarm signal may also be muted for ten minutes.

At the time a measurement is due the acoustic alarm may then be repeated or switched on again. This will also take place after another 10 minutes and 20 minutes. Here, too, the acoustic alarm may be muted for 10 minutes.

After 30 minutes, a further alarm is emitted, which instructs the user to stop the insulin pump, since an automatic mode will leave the system and the insulin pump will thus no longer be automatically stopped. If the operation is continued without measuring the blood glucose level, it is necessary to change to the manual mode of the system. The manual mode involves that the operator sets a new dosage rate for the insulin to be supplied.

Alternatively, an automatic switch-off mode with regard to an automatic stop of the insulin pump may be provided. For this purpose there is a communication link between the insulin pump and the Space Control, so that the Space Control will be enabled to cause the termination of the insulin pump activity. Such an automatic stop mode may be operated as a system which outputs a suggestion to stop the insulin pump, and this system may be manually overridden at any time by the physician or by a nurse who does not want to follow the suggestion, in order to subsequently return to an automatic mode.

If the process is continued not without but with a blood glucose measurement, the operator will input a new glucose value and will set the suggested insulin rate on the insulin pump. Now the system will go back to the automatic mode.

The method according to at least one aspect of the invention for monitoring and regulating blood glucose levels in a blood stream comprises the steps of starting at least one nutrition supply unit for supplying artificial nutrition into the blood stream, determining at least one nutritional value of the artificial nutrition from a plurality of data transferred to a control unit, transferring at least one measured blood glucose value to the control unit and calculating an insulin value and, if applicable, a nutritional value as a function of its effect on blood glucose levels and as a function of the previously measured blood glucose level by means of a calculation unit. Moreover, the calculated new insulin value and, if applicable, any calculated new nutritional values are output by means of an output unit.

The calculated insulin values and, if applicable, the calculated nutritional values may either be transferred in an automated manner to an insulin supply unit and the nutrition supply unit or may be input into the supply units by the operating personnel and, if applicable, be confirmed.

Both of the nutrition units are pumps, with the nutrition supply unit preferably being a pump for an enteral nutrition supply and a pump for a parenteral nutrition supply.

In order to calculate the required insulin values and, if applicable, nutritional values, a patient model is used which takes into account in its calculation model various person-specific data items such as age, the insulin rate administered so far, the body weight and further parameters.

### BRIEF DESCRIPTION OF THE DRAWINGS

Advantages and expediencies will become evident from the following description in conjunction with the drawings, wherein:

FIG. 1 shows a schematic view of a flow chart showing part of the method according to the invention;

FIG. 2 shows a flow chart of a portion of the method according to the invention;

FIG. 3 shows a flow diagram of the method according to the invention;

FIG. 4 shows a flow diagram of a portion of the method according to the invention;

FIG. 5 shows a further illustration of a flow chart of the method according to the invention;

FIG. 6 shows a schematic view of the system according to the invention, and

FIG. 7 shows a time-dependent blood glucose graph for use in the system according to the invention.

### DETAILED DESCRIPTION OF THE PREFERRED EMBODIMENTS

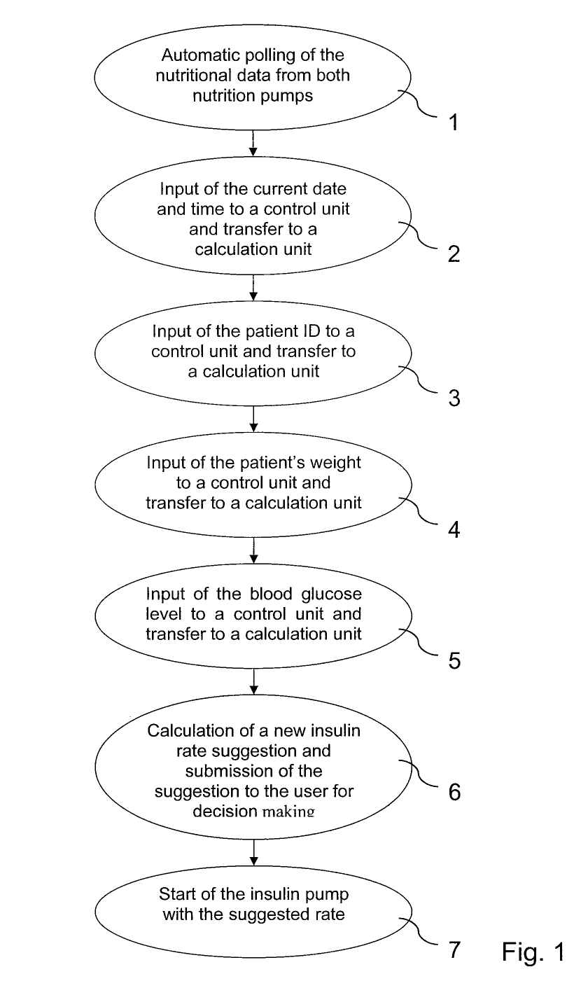

FIG. 1 illustrates a portion of the method according to the invention in a flow chart according to an embodiment of the invention. This is an initialising phase of the method. In such an initialising phase, all of the required data is automated, polled or input manually, at least partially, and a first suggested insulin value is presented. After that, the system according to the invention will wait for further events requiring a new calculation of the insulin rate or an alarm. For example, any nutrition changes, a new blood glucose level, an expiration of the timer for the next blood glucose measurement or a manual change of the insulin rate are to be mentioned.

A control unit (Space Control) 1 sends a polling signal in an automated manner to two nutrition pumps (not shown here in detail), which are used for parenteral and enteral feeding. The polling signal transmits information with regard to the condition of the nutrition pumps and with regard to therapy-relevant data of the type of nutrition present in the pump from the nutrition pumps to the control unit. Especially the carbohydrate value and the current delivery rate are here to be polled as important data with regard to the type of nutrition.

Subsequently or at the same time, an operator will input further data via of the input unit in the form of an input unit such as a keyboard and/or a screen with a mouse. For example, the current time and date are input into the control unit, in order to be transferred to the calculation unit (Space Com) according to step 2. Subsequently, the patient identification number (patient ID) is input into the control unit (Space Control) at 3, in order to be transferred to the calculation unit.

In a further step 4, the patient's weight is input and transferred to the calculation unit.

In step 5, the blood glucose levels are input and transferred.

In step 6, the new insulin rate is calculated in the calculating unit on the basis of the above-indicated values and of the patient model stored in the calculation unit, and this new insulin rate is displayed to the operator on a display unit with a view to a decision to be made. The calculation unit is initiated in an automated way by the control unit, once all the data has been input.

The operator may again evaluate from a medical point of view whether the displayed insulin rate is plausible and may subsequently input this insulin rate into the insulin pump (step 7).

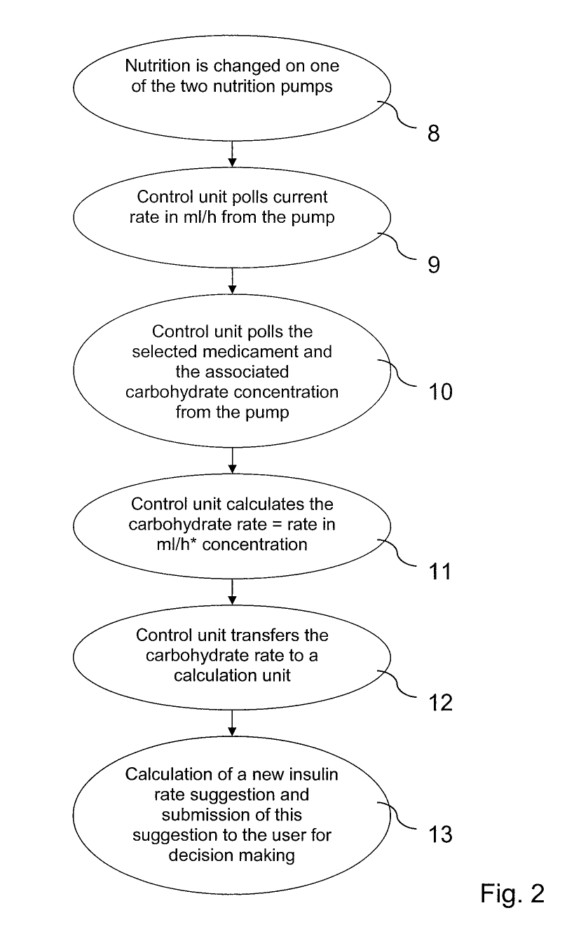

FIG. 2 shows a portion of the method according to the invention as a flow chart. This is the automatic integration of Nutrition and delivered nutritional values upon a change thereof into the calculation of the new insulin value.

According to this, FIG. 1 shows the initial start of the method, whereas FIG. 2 shows the integration of the nutrition, i.e. the response of the blood stream to modifications during an ongoing process.

According to step 8, the type of nutrition and the delivery rate or a further nutrition parameter is changed by the operator on the nutrition supply unit. In such nutrition supply units in the form of nutrition pumps, the nutrition medicament may be selected from the specific nutrition medicament database.

The nutrition medicament data set includes the information about partial concentrations of the nutrition medicament. The nutrition medicament database is created by a separate PC program and is subsequently loaded onto a device in the nutrition pump. For example, a data set for a nutrition medicament may look as follows:

| Name | Nutricomp Standard Neutral |
|---|---|
| Energy | 421 kJ/100 ml |
| Carbohydrates | 13.8 g/100 ml |
| Protein | 3.8 g/100 ml |
| Potassium | 150 mg/100 ml |
| Vitamin A | 90 μg/100 ml |

According to steps 9 and 10, the control unit will cyclically poll the nutrition pump for the delivery rate, the name of the medicament and the carbohydrate concentration. Subsequently, the carbohydrate rate in g/h will be calculated within the control unit in step 11 on the basis of the delivery rate and the concentration and will subsequently be transferred to the calculation unit according to step 12.

In the calculation unit (Space Com), a new insulation rate will be calculated in step 13 and this insulin rate will be presented to the operating personnel on the screen of a display unit.

In response to a change of the relevant input parameters polled in steps 9 and 10, the calculation process will be automatically initiated and a suggestion for a new insulin rate will be calculated. In this connection, the most important relevant input parameters are the blood glucose value, a change of nutrition and/or a change in the patient's weight.

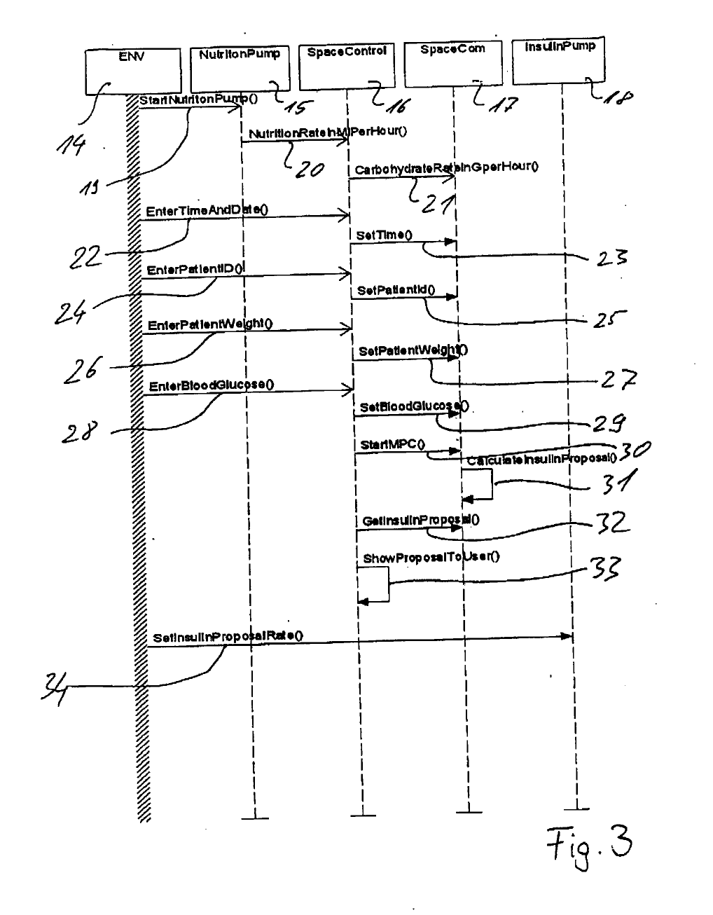

FIG. 3 shows another view of the initialising phase portion of the method according to the invention in the form of a flow chart. The flow chart shown in FIG. 3 illustrates the operator 14, who carries out certain activities with regard to a nutrition pump 15, a control unit 16 and an insulin pump 18. A calculation unit 17 continuously exchanges data with the control unit 16.

In a step 19, the operator 14 starts the nutrition pump 15. In a further step 20, the delivery rate or nutrition rate is delivered by the nutrition pump 15 to the control unit 16.

After that, the control unit 16 sends a carbohydrate rate calculated by it to the calculation unit 17 in a step 21.

In step 22, the current time and date are input into the control unit 16 by the operator. The control unit in its turn forwards this data to the calculation unit in step 23. In the same way, the patient ID is input at 24, the patient's weight is input in a step 26 and the blood glucose level is input into the control unit 16 by the operator 14 in a step 28.

The control unit 16 in its turn transfers the patient ID, the patient's weight and the blood glucose level to the calculation unit 17 in steps 25, 27 and 29.

Subsequently, a suggested insulin value is calculated in a step 13 within the calculation unit 17. After that, the control unit 16 polls the calculation unit for the newly calculated and suggested insulin value in a step 32 and displays this calculated insulin rate on a display unit shown in the control unit 16 in a step 33. If the operator agrees with the suggested insulin rate from a medical point of view, the suggested insulin rate will now be input into the insulin pump 80 by the operator 14 in a step 34.

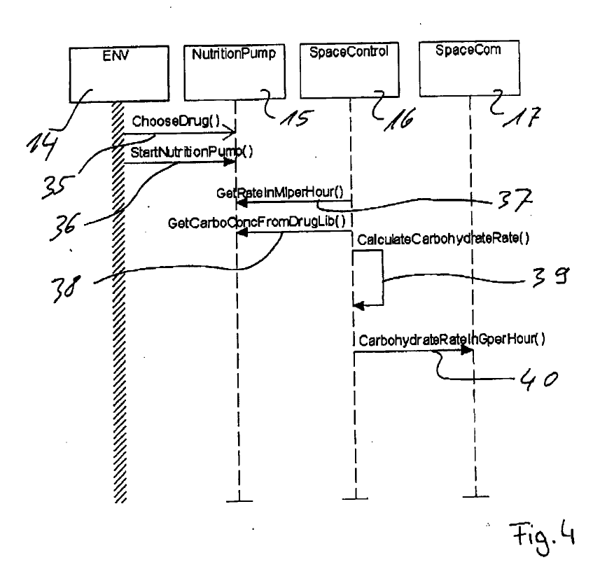

FIG. 4 shows a flow chart of a portion of the method according to the invention. This illustration shows the automatic integration of the nutrition and thus the calculation of the nutritional values into an automated procedure.

In a step 35, an operator 14 selects one of the nutrition medicaments present in the nutrition pump 15. Subsequently, the nutrition pump 15 is started at 36.

In a step 37, a polling signal is sent by the control unit 16 to the nutrition pump 15, in order to obtain the delivery rate or the nutrition rate as information from the nutrition pump 15. Similarly, an input of the carbohydrate concentration for the selected type of nourishment of the artificial nutrition is requested from the control unit 16 in a step 38.

The control unit 16 now calculates in a step 39 a carbohydrate rate from the previous data received in steps 37 and 38 and displays this carbohydrate rate on the control unit and its display unit in a step 40.

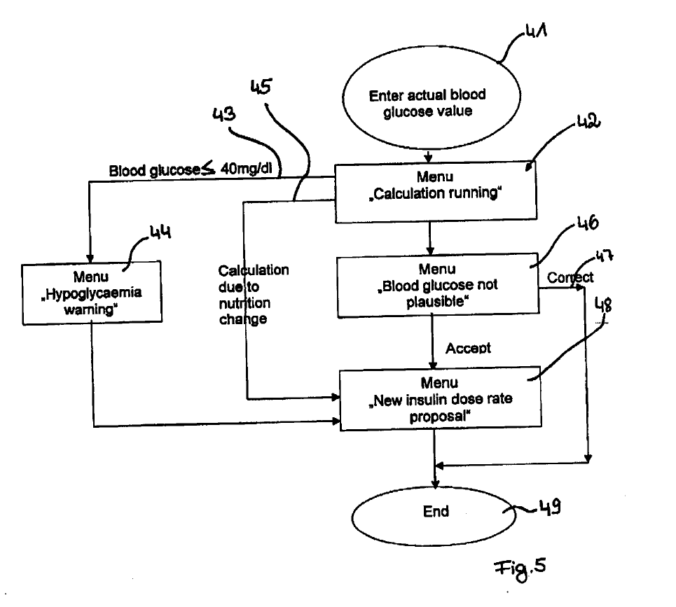

FIG. 5 shows a further schematic flow chart of a calculation procedure for calculating the insulin rate. In step 41, the actual measured blood glucose level, even if this has changed, is input into the system via the input unit.

Subsequently, the calculation unit is initialised by the control unit in a step 42. During the initialising process, “calculation running” is displayed.

If such a calculation process was started as a result of a change in nutrition or a change of the patient's weight, a suggestion for a new insulin rate will immediately be displayed to the operator according to step 45 and step 48. The operator may either set the displayed insulin rate value on the insulin pump or may reject it by inputting a different insulin rate into the insulin pump.

If the calculation mode was started as a result of a newly measured blood glucose level, a hypoglycaemia warning is indicated to the operator in steps 43 and 44, if the newly input blood glucose level is below 40 mg/dl.

If the input blood glucose level does not fall below such a value, which may also be different to the one shown, a type of plausibility check of the input value with regard to the further values present in the calculation unit and as a function of an underlying patient model will be carried out in a step 46.

If the plausibility check shows that the input value does not appear to be plausible in the light of the further values, this step 46 will be shown on the display. Depending on the displayed value, the operator will now have to make a decision as to whether a measurement error may be present, so that the measurement has to be discarded, in order to carry out a new measurement, or whether this blood sugar value is correct and is therefore admitted in step 47.

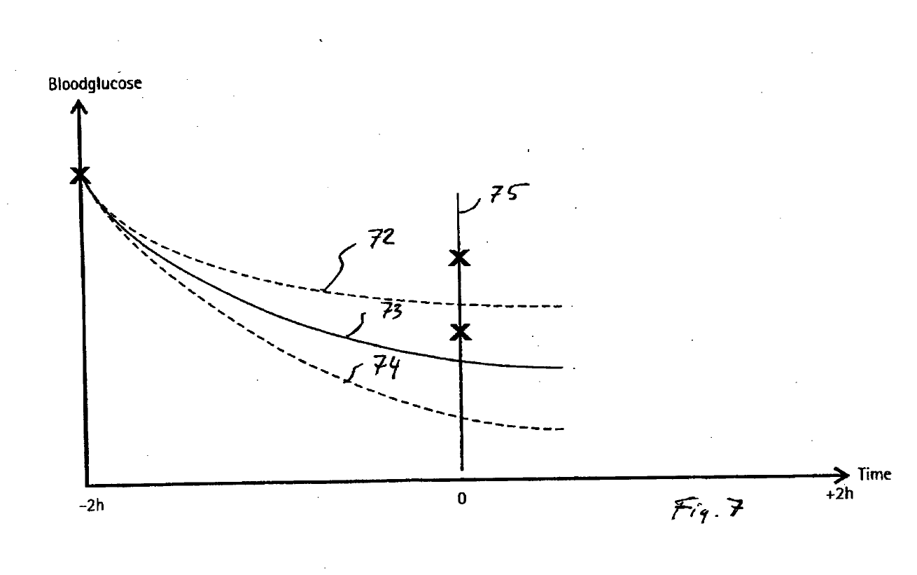

A plausibility check will then be carried out in a procedure that will be illustrated in more detail in the diagram shown in FIG. 7. FIG. 7 shows a graph of the expected blood glucose value curve over the next four hours. At that point in time, a trajectory will be calculated on the basis of the blood glucose value to be expected. This corresponds to curve 73. In the same way, any expected cone-shaped error areas will be calculated and established via an upper and a lower limit according to curves 72 and 74 for the blood glucose level. If after two hours within this area cone, the measured values, which are on a line 75, are between the lines 72 and 74, the measured value appears to be plausible. Any measured values lying outside of the area cone between the lines 72 and 74 will be indicated to the operator as a warning.

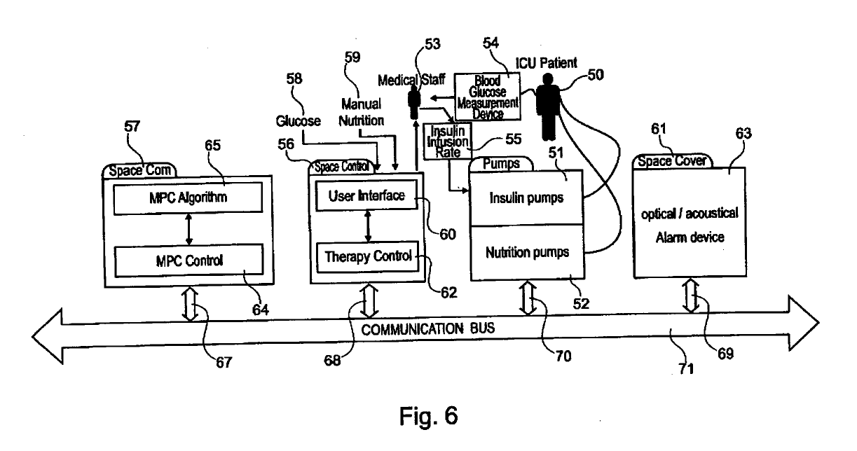

FIG. 6 shows a schematic view of an embodiment of the system according to the invention. The operator 53 receives blood glucose values measured by means of a blood glucose measurement unit 54. In the same way, the operator 53 may transfer insulin rates 55 to or input them into an insulin pump 51, and, if applicable, may also input nutritional values into the nutrition pump 52. Both pumps 51 and 52 are connected to a patient 50.

A control unit 56 is in direct communication with a calculation unit 57 via a common data line 71, which is schematically shown here.

A blood glucose level 58 and a manual input of nutritional values 59 takes place via an input unit 60, which is at the same time an output unit. The input unit 60 is in direct communication with a therapy control unit 62 which is responsible for the exchange of data and the control and regulation thereof. Consequently, secure and unsecure communication data is exchanged between the control unit 56 and the calculation unit 57 as well as the pumps 51, 52 and an alarm unit 63 via several channels 68, 69, 70 and 67.

An alarm signal is generated in the visual and/or acoustic alarm unit 63, if a new measurement of the blood glucose value upon expiration of a measurement time interval is due to be taken or if a suspension of the supplied insulin rate and/or a suspension of any supplied nutritional values and/or a great change of the blood glucose level occurs. This may be indicated both by a visual and an acoustic alarm, in order to alert any personnel who may presently not be on site.

In the calculation unit 57, a calculation of the insulin rate and, if applicable, of further data such as new nutritional values is carried out by means of calculation programs 65 stored therein and an MPC controller 64 cooperating therewith.

All of the features disclosed in the application documents are claimed as being essential to the invention, in as far as they are novel over the prior art either individually or in combination.

1-19. (canceled)

20. A system for monitoring and regulating blood glucose levels in a blood stream, comprising:
an input unit for receiving at least one measured blood glucose level present in the blood stream, at least one insulin value so far supplied to the blood stream by means of at least one insulin supply unit, and/or at least one nutritional value of nutrition so far directly or indirectly supplied to the blood stream by means of at least one nutrition supply unit,
a calculation unit for calculating a new insulin value and/or a new nutritional value as a function of its effect on the blood glucose levels and as a function of the previously measured blood glucose level, and
an output unit for outputting the new insulin values and/or nutritional values.

21. The system as claimed in claim 20, wherein the input and the output unit are combined in a control unit with a transmission and a reception unit.

22. The system as claimed in claim 20, wherein the output unit is connected to a display unit.

23. The system as claimed in claim 20, wherein the output unit is connected to the nutrition supply unit.

24. The system as claimed in claim 20, wherein the output unit is connected to the insulin supply unit.

25. The system as claimed in claim 21, further comprising at least one polling signal which is sent by the control unit for polling the nutritional values to the nutrition supply unit at least at one predeterminable point in time.

26. The system as claimed in claim 25, wherein the polled nutritional values comprise the current delivery rate of the nutrition supply unit embodied as a pump, the type of nutrition and the carbohydrate concentration thereof.

27. The system as claimed in claim 20, wherein the input unit includes an input unit for inputting the measured or calculated blood glucose levels and/or nutritional values and further person-specific values such as body weight, age of the person and such data.

28. The system as claimed in claim 20, further comprising an alarm unit for emitting a visual or acoustic alarm to an operator indicating that it is time for the measurement of the blood glucose level in the blood stream.

29. A method for monitoring and regulating blood glucose levels in a blood stream, comprising the following steps:
starting at least one nutrition supply unit for directly or indirectly supplying nutrition into the blood stream,
determining at least one nutritional value of the nutrition from a plurality of data transferred by the nutrition supply unit to a control unit,
transferring measured blood glucose levels present in the blood stream to the control unit,
calculating an insulin value and, if applicable, a nutritional value as a function of its effect on blood glucose levels and as a function of the previously measured blood glucose level by means of a calculation unit, and
outputting the calculated new insulin values and, if applicable, the calculated new nutritional values by means of an output unit.

30. The method as claimed in claim 29, wherein the output calculated insulin values are transferred to an insulin supply unit or input therein.

31. The method as claimed in claim 29, wherein the output calculated nutritional values are transferred to the nutrition supply unit or input therein.

32. The method as claimed in claim 29, wherein the step of determining the nutritional value includes the following:
polling a delivery rate, the type of nutrition and a carbohydrate concentration from the nutrition supply unit by the control unit,
calculating the carbohydrate rate as nutritional values from the delivery rate, the type of nutrition and the carbohydrate concentration by means of the control unit,
transferring the carbohydrate rate to the calculation unit for calculating new insulin values and, if applicable, new nutritional values.

33. The method as claimed in claim 29, wherein person-specific data is input into the control unit for calculating the new insulin values.

34. The method as claimed in claim 29, wherein the new nutritional values are calculated when the nutrition supply unit interrupts the nutrition supply or changes its delivery rate.

35. The method as claimed in claim 29, wherein an operator is alerted by means of an alarm unit disposed in the control unit to indicate that it is time for the next measurement of the blood glucose level in the blood stream.

36. The method as claimed in claim 35, wherein the operator is alerted by means of the alarm unit in the case of a change to the supplied nutritional values, which was not predetermined.

37. The method as claimed in claim 29, wherein the values output by the calculation unit are displayed to an operator and confirmed by the operator as applicable.

38. The method as claimed in claim 29, wherein a plausibility check with regard to the plausibility of the measured blood glucose level is carried out.
